(19) United States
(12) Patent Application Publication
(10) Pub. No.: US 2024/0000595 A1
(43) Pub. Date: Jan. 4, 2024

(54) POLYMERIC POLYCENTRIC HINGE

(71) Applicant: Ossur Iceland ehf, Reykjavik (IS)

(72) Inventors: Halldor ALBERTSSON, Reykjavik (IS); David SANDAHL, Reykjavik (IS); Valgeir PETURSSON, Reykjavik (IS)

(21) Appl. No.: 18/342,859

(22) Filed: Jun. 28, 2023

Related U.S. Application Data
(60) Provisional application No. 63/356,698, filed on Jun. 29, 2022.
(60) Provisional application No. 63/439,513, filed on Jan. 17, 2023.

(51) Int. Cl. A61F 5/01

(52) U.S. Cl. CPC A61F 5/0123; A61F 2005/0139

(57) ABSTRACT

A polycentric hinge for an orthopedic device includes first and second hinge arms, and two cover plates, rather than numerous individual parts and fasteners. The first and second cover plates define a plurality of bearings and a plurality of recesses for receiving the bearings, whereby the hinge arms are mounted about the bearings and are pivotable relative to one another while encased by the first and second cover plates. Additional bearing surfaces are provided for securing snap-fitted rotation stops without a tool or fasteners. The components of the hinge are formed from injection molded materials such as plastics.

### FIELD OF THE DISCLOSURE

This disclosure relates to a polycentric hinge in an orthopedic device, preferably formed by plastic, for supporting, limiting, or controlling joint movement, including removable and interlocking stops to modify the range of motion of the hinge.

### BACKGROUND

Many orthopedic devices include hinges arranged to support joints and control and limit joint movements. These joints include the knee, elbow, shoulder, hip, ankle, and wrist joints.

The knee, although frequently considered a hinge joint, comprises two joints, lateral and medial, between the femur and tibia and one arthrodial joint between the patella and femur. The primary movements of the knee comprise flexion (i.e., rearward rotational movement of the tibia relative to the femur) and extension (i.e., the knee is straight, and the angle between the femur bone and the tibia bone is generally at 0 degrees).

The flexion and extension movements of the knee joint are not simply pivotal movements about a fixed axis. During flexion, the axis around which movement occurs shifts backward, and during extension, movement shifts forward. This movement differs from a typical hinge joint, such as an elbow, where the axis of rotation typically does not shift. As full extension is reached, the tibia is rotated inward or rearward, and the joint is oriented in a “locked” position with the ligaments taut. This arrangement gives the joint greater stability in the extended position. As flexion is initiated, the tibia initially lowers or moves downward with a small external rotation of the tibia, unlocking the joint. Then the tibia rotates or rolls about the joint to full flexion. The initial unlocking of the knee joint during flexion precedes actual full knee rotation.

Because of the complexity associated with knee movement, a knee brace hinge mechanism should be able to simulate knee movements. Incorporating such a hinge mechanism is important, as the knee brace must optimally support the knee joint of its user.

In post-surgical applications, the requirement for such simulation of the knee joint is important to rehabilitate and prevent re-injury of an injured knee joint. In recognizing the need for an effective post-surgical knee brace, various hinge mechanisms have been incorporated into known knee braces for supporting and controlling knee movement. One type of hinge used for duplicating movement of the human knee joint in an orthopedic device is a plural axis or polycentric hinge. As taught in U.S. Pat. Nos. 4,524,764, 4,732,143, and 5,443,444, a polycentric hinge is used to support the knee joint throughout its full range of motion or to lock the knee joint in a selected position or to limit the allowed range of motion of the joint to less than complete extension (straightening) and or less than complete flexion (bending) of the knee.

U.S. Pat. No. 9,668,903, granted Jun. 6, 2017, and incorporated herein by reference in its entirety, discloses a polycentric hinge for an orthopedic device having first and second hinge arms and two cover plates rather than numerous individual parts and fasteners. The first and second cover plates define a plurality of bearings and a plurality of recesses for receiving the bearings, whereby the hinge arms are mounted about the bearings and are pivotable relative to one another while encased by the first and second cover plates. Tools and fasteners are required to secure flexion and extension stops to the cover plates.

Because of (i) the complex repeat movement of the knee, (ii) the need to support the knee through a range of motion, and (iii) the ability to lock or restrict knee movement, existing polycentric knee brace hinges are complex and may include numerous parts, making such devices complex and expensive. In addition, while there are many solutions for providing and securing flexion and extension stops, prior art solutions require tools, multiple parts to the hinge, and complicated methods for installing and removing flexion and extension stops.

It would be an advance within the art to provide a polycentric hinge of reduced components without detracting from the beneficial functionality associated with existing polycentric hinge structures.

### SUMMARY

In an embodiment of the present disclosure, a polycentric hinge for an orthopedic device comprises a first hinge arm including a hole at a geared end thereof, a second hinge arm including a hole at a geared end thereof, and first and second cover plates for hingedly joining the first and second hinge arms with no intermediate plates or other elements. The first and second cover plates define a plurality of protruding tubular bearings and corresponding openings (e.g., two recesses) for receiving cooperating protrusions to secure the hinge together. The bearings and protrusions may be oppositely located relative to one another to allow each bearing to be received into corresponding openings when the cover plates are brought together, e.g., positioned over and aligned relative to one another. The hinge plates are configured to interlock, become integrally connected, and be permanently secured to one another without fasteners.

In a preferred embodiment, the hinge is arranged to avoid fasteners or metal parts. The components of the hinge, the first and second hinge arms, and the first and second cover plates, in a preferred embodiment, can consist solely of minimal parts, such as four parts excluding the flexion and extension parts. Consistent with being formed by few or a minimum of parts, the parts may be formed from the same or compatible polymeric materials (e.g., materials capable of being chemically bonded or ultrasonically welded). In addition, the parts may be secured without connective bolts, nails, soldering materials, or adhesives to bind the materials together.

Due to the simplicity of using fewer parts, the parts may be molded by injection molding, by shaping liquid, or pliable raw material using a rigid frame called a mold or matrix. The shape and structure may be made using a pattern or model of the final object. The raw material is preferably plastic, allowing for repeatable and multiple parts to be made at minimal cost, as opposed to prior art hinges that mix metal and plastic components. The plastic material may be reinforced with carbon, glass fiber, or other suitable reinforcing fibers to provide strength, and some parts may be reinforced, such as the hinge arms, whereas the cover plates may not be reinforced. However, such a selection may be made according to implementing the hinge in a given orthopedic brace. The parts need not be made from the same material.

In an embodiment of the cover plates, a first surface of a first cover plate may be arranged to extend flush with the top surface of the first and second bearings of an interior side of a second cover plate to minimize or eliminate fasteners. First and second protrusions extending from the first cover plate may have a length greater than the combined length of the first and second bearings and a thickness of the second cover plate such that an excess portion of the first and second protrusions extends beyond an exterior surface of the second cover plate.

The excess portion is arranged to be melted or sonically welded to fill an outlet recess formed along an exterior surface to secure the first cover plate to the second cover plate. Depending on the first and second openings, the outlet recess may have an enlarged diameter relative to accommodate the excess portion being melted by welding or other appropriate techniques to form an integral connection of the first cover to the second cover. In addition, the polymeric material from the first and second covers may mix to form a cohesive bond and material section.

The first and second cover plates are preferably chemically and integrally bonded. The excess portion and the material of the second cover plate at the cohesive bond are inseparable and form a continuous structure bonded to act mechanically as a monolithic structure. By chemical and integral bonding, a preferred embodiment is without an adhesive. During bonding or welding, the excess portion and sections of the second cover plate are sufficiently fluid for the materials to at least slightly blend into one another at an interface.

The extension of the excess portion protruding from the exterior surface of the second cover plate is advantageous over known hinges in that the excess portion can be manipulated and visualized to bond or weld to the second cover plate.

At least one of the cover plates includes at least one of the bearings having coaxial openings, and the other of the cover plates includes protrusions insertable into the openings of the bearings. The same cover plate may form both bearings, and the protrusions may extend from the other cover plate such that the height of the bearings forms a clearance between the first and second cover plates once the protrusions are inserted into the openings of the bearings. Alternatively, each cover plate may include one bearing and one protrusion. The hole of one hinge arm is positioned over its corresponding bearing. In contrast, the hole of the other hinge arm is positioned over another bearing corresponding to that hinge arm so that when the cover plates are positioned, they are one over the other.

The cover plates preferably encase the geared ends of the hinge arms, maintaining the hinge arms in orientation, so gear teeth at the geared ends mesh together, hingedly connecting each of the first and second hinge arms to one of the bearings. As mentioned above, the first and second cover plates may be secured together. For example, the cover plates may be formed of a polymeric material. The cover plates may be secured together using pulse staking, a form of heat staking, with the bearings passing through the holes of the hinge arms and into the recess of the opposite cover plate, holding the hinge arms meshed, with each arm being hingedly connected to the cover plates such that they are hingedly rotatable about the bearing received through the hole of the hinge arm.

Advantageously, the bearings defined by the cover plates themselves do not necessitate additional fasteners for securing the hinge arms to the cover plates. The construction of the bearings defined by the cover plates themselves removes a requirement for additional components (i.e., fasteners or bearings) while providing a more streamlined shape to the polycentric hinge, particularly by encasing all components of the polycentric hinge between the first and second cover plates, as opposed to known polycentric hinges that include metal fasteners extending through the components of the hinge.

In another advantageous hinge construction, the flexion and extension stops can be removably secured to the hinge with no fasteners. Additional bearings are formed by the cover plates that accommodate directed fastening of stops without additional fasteners. For example, the stops may form resilient prongs that can snap-fit to the additional bearings such that the stops are positioned to limit the rotation of the hinge arms. Such an arrangement permits an easy range of motion adjustment in-field use using no tool. In addition, the stops can fasten or snap-fit to the additional bearings, and, with additional effort beyond the normal use of the hinge, a clinician or user can remove the stops or replace the stops with other stops according to a different range of motion for each flexion or extension (typically defined in degrees).

In constructing the polycentric hinge of the embodiments of the disclosure, the outer surfaces of the cover plates are substantially smooth without interruption by any additional features, as in extraneous fasteners in the prior art. Weight is reduced since the cover plates of the polycentric hinge are preferably solely constructed from a polymeric material. Greater flexibility in adapting the polycentric hinge to various orthopedic devices is enabled since the shape and robustness can be modified by selecting polymeric materials for only the cover plates without considering non-polymeric material components or other components required for making the hinge in the prior art polycentric hinges.

According to another embodiment, the polycentric hinge consists of a first hinge arm including a hole at a geared end, a second hinge arm including a hole at a geared end, and first and second cover plates for hingedly joining the first and second hinge arms together. The cover plates encase the geared ends of the first and second hinge arms, maintaining the hinge arms in orientation, so gear teeth at the geared ends of the first and second hinge arms mesh together, hingedly connecting the first and second hinge arms to the bearings. The first and second cover plates are preferably permanently secured together.

The bearings maintain the hinge arms orientation, so gear teeth at the geared ends of the upper and lower hinge arms mesh together as the hinge arms are rotated about their respective bearing during use. Finally, the cover plates are brought together and positioned over one another to encase the geared ends of the hinge arms between the inner and outer cover plates, hingedly connecting the upper and lower hinge arms to a respective one of the bearings.

The disclosed orthopedic devices and associated methods of manufacture provide a simpler polycentric hinge, formed from just two parts (e.g., first and second cover plates) for joining the first and second hinge arms, which allows assembly of the polycentric hinge to be achieved faster, in a less complex manner, and with less expense. In addition, as shown, the polycentric hinges (e.g., formed of nylon or another suitable polymeric material) have been found to exhibit strength and durability characteristics equal to or better than existing polycentric hinges formed of numerous (e.g., metal) components.

Numerous other advantages, features, and functions of embodiments of a polycentric hinge will become readily apparent and better understood because of the following description and accompanying drawings.

### Glossary

As used, the term “posterior” has its ordinary meaning and refers to behind or to the rear of a joint or facing towards the user's body. Likewise, the term “anterior” has its ordinary meaning and refers to a location ahead of or the front of a joint or facing away from the user's body.

The terms “rigid,” “flexible,” and “resilient” may distinguish characteristics of portions of certain features. The term “rigid” should denote that an element generally lacks flexibility. Within the context of features that are “rigid,” it should indicate that they do not lose their overall shape when force is applied and may break if bent with sufficient force. The term “flexible” should denote those features capable of repeated bending such that the features may be bent into retained shapes or retain no general shape but continuously deform when force is applied. The term “resilient” should denote an element or feature that is not easily broken and may withstand continued use or movement.

The term “user” refers to a person who uses the hinge in an orthopedic device. The user may be a patient or an operator. The term “clinician” refers to a clinical specialist, supervisor, therapist, doctor, or person with a similar role that assists or oversees the operation of the device, including the hinge, as in an orthopedic device, by the user.

The term “plurality” connotes two or more of a given element or feature.

The term “outer” means the cover plate is away from the user's body. Likewise, the term “inner” is intended to mean close or proximate to the body of the user, with the plate located on the opposite side from the inner plate.

While terms such as “first” and “second” may be used in combination with terms such as “upper” and “lower” or “inner” and “outer,” the terms “first” and “second” may be used interchangeably with such terms “upper” and “lower,” or “inner” and “outer,” and are not necessarily limited to being associated with such terms.

Similarly, the terms “third” and “fourth” may be used in combination with terms such as “anterior” and “posterior” or, when in reference to stops, may be used similarly to “first” and “second” in combination with terms such as “upper” and “lower” or “inner” and “outer.”

The term “interior” refers to the surface or area facing the inner working of the hinge, which is not seen unless the hinge is opened.

The term “bearing surface” has its ordinary meaning and refers to the contact area between two elements.

The term “hinge arm” refers to the elements extending away from the hinge mechanism, which may be used to attach the hinge to an orthopedic brace.

The term “geared ends” refers to the interior end of the hinge arms, which includes gear prongs, fitted together, and allowing for rotation within the hinge.

The term “protrusion” has its ordinary meaning and refers to an element extending from a surface.

The terms “flexion” and “extension” refer to the position of the hinge or joint of the user while the hinge is in use. The term “flexion” is intended to mean bending of the hinge or joint, and in the example of the knee, rearward rotational movement of the tibia relative to the femur. The term “extension” refers to straightening the joint or hinge. In the example where the knee is the joint in use, “extension” refers to when the angle between the femur bone and the tibia bone is generally at 0 degrees,

The term “cover plate” refers to the exterior element covering the hinge mechanism and the surface upon which additional features may be built inside the hinge.

The term “stops” refers to elements that halt movement and limit the extent of “flexion” or “extension” rotation.

The term “peripheral” has its ordinary meaning and refers to an element's edge or surrounding surface.

The term “clearance” refers to the space, distance, or allowance between elements.

The term “plastic” may be used interchangeably with polymeric materials, although plastic is a type of polymeric material otherwise known as being comprised of a chain of polymers.

The term “plastic” does not negate the possibility of reinforcements to the plastic material. The term “reinforcement” is used to denote materials that can enhance the mechanical properties of plastic and embrace different types of fibers and a variety of other materials to modify the mechanical properties of the plastic.

The term “interlock” describes two or more components engaging with each other by overlapping or fitting together of projections and the like, and recesses and the like.

The term “heat staking” refers to a known process of using local heating and cooling to raise the temperature of plastic components and allow plastic reforming to be carried out.

The term “ultrasonic welding” refers to using high-frequency ultrasonic acoustic vibrations applied locally to workpieces to hold them together under pressure to create a solid-state weld.

The term “reinforcement” or “reinforcing” fibers refers to synthetic fibers such as glass, carbon, graphite, and aramid that are available as short, long, fabric, or woven reinforcement in thermosets and thermoplastics. The inclusion of reinforcement fibers significantly reduces wear and improves mechanical properties over thermosets and thermoplastics without such reinforcement fibers.

It will be understood that unless a term is defined to possess a described meaning, there is no intent to limit the meaning of such term, either expressly or indirectly, beyond its plain or ordinary meaning. The embodiments of the disclosure are adapted for a human body and may be dimensioned to accommodate different types, shapes, and sizes of human body sizes and contours.

### BRIEF DESCRIPTION OF THE DRAWINGS

The drawing figures are not necessarily drawn to scale but instead are drawn to provide a better understanding of the components thereof and are not intended to be limiting in scope but provide exemplary illustrations. The figures illustrate exemplary configurations of an orthopedic device and in no way limit the structures or configurations of a simplified polycentric hinge according to the present disclosure.

FIG. 1 is a perspective view of an embodiment of a hinge.

FIG. 2 is an exploded view of the hinge in FIG. 1.

FIG. 3 is a plan view of the hinge in FIG. 1 without a first cover plate, including flexion and extension stops secured to the hinge.

FIG. 4 is a cross-sectional elevational view of the hinge in FIG. 1 along line IV-IV.

FIG. 5 is a perspective view of the first cover plate of FIG. 1.

FIG. 6 is a plan view of the second cover plate of FIG. 1

FIG. 7 is a cross-sectional elevational view of another embodiment of the hinge in FIG. 1.

FIG. 8 is a perspective view of the first cover plate of FIG. 7.

FIG. 9 is a perspective view of the second cover plate of FIG. 7.

FIG. 10 is a plan view of another embodiment of the hinge in FIG. 1 with alternate flexion and extension stops disassembled from the hinge, with the stops on the anterior side being alternately or selectively used but not used simultaneously in an application of the hinge.

FIG. 11 is a plan view of the embodiment of the hinge in FIG. 10 with 0 degree and 90 degree flexion stops attached to the hinge.

### DETAILED DESCRIPTION OF VARIOUS EMBODIMENTS

### A. Overview

A better understanding of different embodiments of the disclosure may be had from the following description read with the accompanying drawings in which reference characters refer to like elements.

While the disclosure is susceptible to various modifications and alternative constructions, certain illustrative embodiments are in the drawings and are described below. It should be understood, however, that there is no intention to limit the disclosure to the specific embodiments disclosed, on the contrary, the intent is to cover all modifications, alternative constructions, combinations, and equivalents falling within the spirit and scope of the disclosure.

### B. Various Embodiments of a Polycentric Hinge

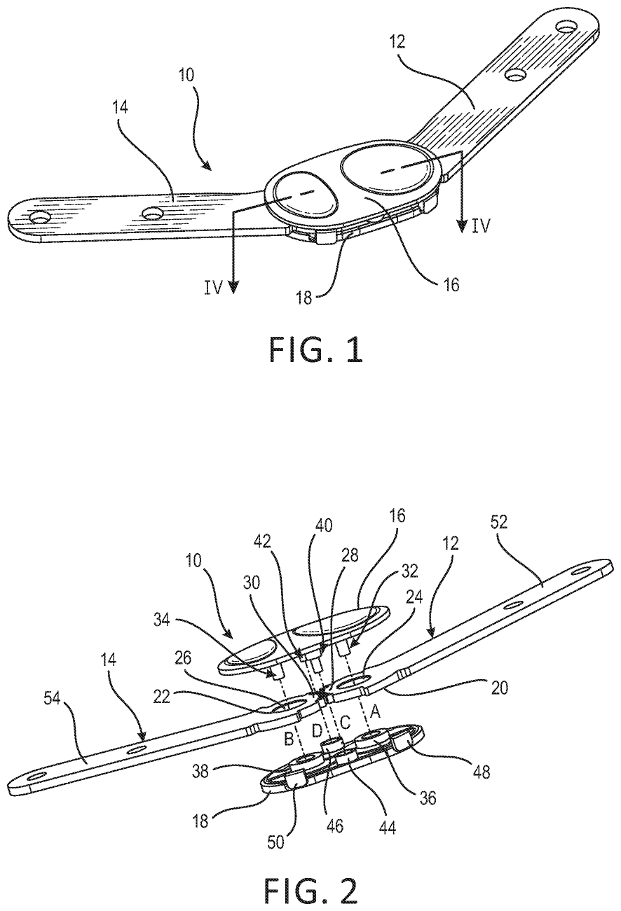

FIGS. 1-6 illustrate an exemplary embodiment of an orthopedic device (e.g., a knee brace) according to the present disclosure that exhibits increased simplicity, streamlined shape, and reduction in weight, where the hinge comprises only first and second cover plates 16, 18 that may advantageously be welded (e.g., pulse staking) together and supports first and second hinge arms 12, 14. Furthermore, these four components, the first and second cover plates 16, 18, and the first and second hinge arms 12, 14, are preferably formed by a polymeric material or plastic and may be molded, such as by injection molding.

By molding the parts of the hinge, particular advantages are obtained. A large volume of parts can be quickly produced, and small parts of the components, such as holes or apertures, are formed by the material in a single molding process instead of being machined or in post-production processes. The components may be ready as molded without requiring further finishing steps. Tight or small dimensions may be obtained, such as with gear teeth. The components are monolithically formed and consist of a single material, or set of materials, consistently across the structure of the component. These features contrast with prior art hinges that may comprise components formed from dissimilar materials.

The polycentric hinge 10 comprises solely two cover plates, including a first or outer cover plate 16 and a second or inner cover plate 18, in which such cover plates form all features enabling such cover plates to be secured together by bonding, ultrasonic welding, or by pulse staking (a form of heat staking) and for supporting the hinge arms. It is by such molded structure formed in the molding process and the material of the cover plates that enable them to perform their intended purpose.

The first hinge arm 12 defines a first hole 24 at a first geared end 20, and the second hinge arm 14 defines a second hole 26 at a second geared end 22. The first and second hinge arms 12, 14 include struts 52, 54 extending therefrom for securing to an orthopedic device. The first and second geared ends 20, 22 define the first and second geared profiles 28, 30, arranged to mesh and articulate relative to one another.

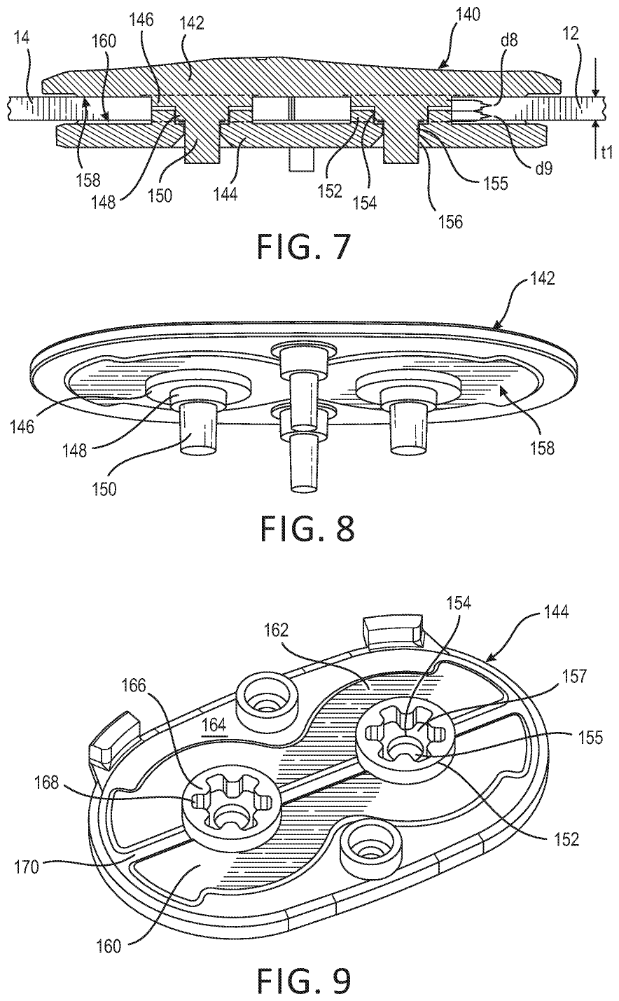

The first and second cover plates 16, 18 hingedly join the first and second hinge arms 12, 14. The first cover plate 16 defines first and second protrusions 32, 34 extending from a bearing surface 138 protruding from an interior first surface 33 thereof, which is arranged to extend flush with a top surface 37. The second cover plate 18 defines first and second tubular bearings 36, 38 protruding from a bearing surface 122, which protrudes from an interior first surface 35 of the second cover plate 18. The bearings 36, 38 are arranged for receiving the first and second protrusions 32, 34 into the first and second openings 124, 126. The first and second bearings 36, 38 extend a predetermined distance d1 at least a length at or greater than a thickness t1 of the first and second hinge arms 12, 14. In an alternative embodiment shown in FIG. 7, the protruding bearing surface 158 on the interior first surface 142 of the second cover plate 18 may additionally be designed with a midline 140 to minimize the effect of the knit line (bump) that appears as an uneven surface where the plastic meets after flowing around the first and second tubular bearings 144, 146.

The first and second holes 24, 26 of the first and second hinge arms 12, 14, respectively, are sized and configured with a diameter d3 at least a size of an outer diameter d4 of the first and second bearings 36, 38 to freely rotate about the first and second bearings 36, 38. The first and second holes 24, 26 are coaxial to the first and second bearings 36, 38. The first and second protrusions 32, 34 extend coaxially relative to the first and second bearings 36, 38 and the first and second holes 24, 26 along first and second axes A, B.

Upon assembly of the cover plates 16, 18 about the hinge arms 12, 14, the geared ends 20, 22 of each hinge arm 12 and 14 become sandwiched or encased between cover plates 16, 18. The hinge arms are preferably formed by polymeric materials or a combination of polymeric material and reinforcing fibers, such as carbon, glass, or any other reinforcing type of fibers. The hinge arms may be rigid or semi-rigid or flexible, whereby the material composition and relative rigidity of the hinge arms depends upon the usage of the hinge.

The first and second bearings 36, 38 are spaced apart relative to one another so the geared profiles 28, 30 at the geared ends 20, 22 of each hinge arm 12, 14 can mesh together. Because of such a meshed arrangement, rotation of one hinge arm about its respective bearing (e.g., arm 12 about bearing 36) results in complementary rotation of the other hinge arm about its respective bearing (e.g., arm 14 about bearing 38). The bearings 36, 38 themselves serve the dual purpose of pivot pins for the hinge arms and receptacles for receiving protrusions of the opposing cover plate to interlock and secure the cover plates and the components of the hinge together while containing all geared ends 20, 22 of the hinge arms 12, 14 together in a fixed configuration. As a result, the outer surfaces of the first and second cover plates may be substantially smooth with no extraneous components projecting therefrom, as may occur in prior art polycentric hinges.

The first surface 33 of the first cover plate 16 is arranged to extend flush with a top surface 37 of the first and second bearings 36, 38. The first and second protrusions 32, 34 have a length greater than a combined length of the predetermined distance d1 of the first and second bearings 36, 38 and a thickness t2 of the second cover plate 18 such that an excess portion 64 of the first and second protrusions 32, 36 extends beyond the second cover plate 18.

The excess portion 64 is arranged to be melted or sonically welded to fill an outlet recess 58 formed along an exterior surface 39 to secure the first cover plate 16 to the second cover plate 18. The outlet recess 58 depends on the first and second openings 124, 126, and has an enlarged diameter relative thereto to accommodate molded flashing melted thereinto by welding or other appropriate techniques to form an integral connection of the first cover to the second cover whereby polymeric material from the first and second covers mix with one another to form a cohesive bond and material section.

The first and second protrusions 32, 34 each form a base portion 60 with a base width or diameter, a base length d6, and an extension portion 62 extends from the base portion 60. The base width or diameter is greater than an extension width or diameter. The extension portion 62 includes the excess portion 64. The base length d6 generally corresponds to the predetermined distance d1, and the extension portion has a length d7 exceeding the thickness t2 of the second cover plate 18. The first and second openings 124, 126 define an opening base portion 132 configured and dimensioned to the base portion 60. The base portion 60 rests along a periphery 56 defines by the transition of the opening base portion 132 to the opening extension portion 134.

The hinge 10 may include components and structural features similar to existing polycentric hinges, but only in which any such features may be integrated within the molded or otherwise formed two cover plates. Unlike in the prior art, the second cover plate 18 defines third and fourth tubular bearings 44, 46 located between and offset from the first and second bearings 36, 38, which are configured and dimensioned to retain flexion and extension stops 88, 86, which can be snap-fit to the third and fourth bearings 44, 46 to limit rotation of the hinge arms relative to each other. The third and fourth bearings 44, 46 are preferably located proximate a peripheral edge 21 of the second cover plate 18 and protrude from the second cover plate 18, and are parallel to the first and second bearings 36, 38. The third and fourth bearings 44, 46 are preferably located proximate to a peripheral edge 19 of the first cover plate 16 and preferably have a length the same as the predetermined distance d1 of the first and second bearings 36, 38.

The third and fourth bearings 44, 46 define an outer diameter d5 that is preferably smaller than the outer diameter of the first and second bearings 36, 38 so as not to interfere with rotation of the hinge arms, particularly with the geared ends of the hinge arms.

Similar to the first and second protrusions, yet dimensioned accordingly, the first cover plate defines third and fourth protrusions 40, 42 arranged to extend into the third and fourth openings 128, 130 in the third and fourth tubular bearings 44, 46 along the third and fourth axes C, D. The third and fourth protrusions 40, 42 are arranged similarly with a base portion and an extension portion, as in the first and second protrusions 32, 34, and the third and fourth openings 128, 130, including the third and fourth protrusions 40, 42 having an excess portion used to bond and form an integral connection of the first cover plate 16 to the second cover plate 18, as in the excess portion of the first and second protrusions 32, 34. The excess portion of the third and fourth protrusions 40, 42 may extend to the same degree as the excess portion 64, or differently. Likewise, the third and fourth openings 128, 130 may provide an outlet recess as in the first and second openings 124, 126.

To get a consistent outcome of hinge thickness and avoid the hinge having a too loose or too stiff assembly, protrusions 32, 34, 40, 42 have been shortened d2 so that they never come to a hard stop, but the plates 16, 18 come to a hard or full stop with the hinge arms 12, 14. The hinge stiffness is controlled by fixed pressure applied to the assembly while welding.

It will be noted that the cover plates are not limited to having the bearings and protrusions. For example, the first cover plate 16 may define the bearings, the second cover plate 18 may define the protrusions, or each cover plate may include some bearings and protrusions, and the other cover plate is configured accordingly.

The cover plates may be of similar or identical shape, to provide a profile generally coextensive one with the other when the cover plates are placed over one another. As shown, the profile of each cover plate may be oval, so upon placement of the two cover plates together, they are generally coextensive with one another. Because of the molded nature of the cover plates, they may be shaped and scaled according to a desired application of the hinge in an orthopedic device. For example, the general features of the hinge may be scaled down in an elbow hinge, yet scale more robustly for a knee brace, particularly if any weight-bearing is involved. Thus, the molds can be made according to the application while preserving all functional and structural features. Alternatively, one of the cover plates may be larger in surface area than the other. When the cover plates are oval, one of the cover plates may be a slightly larger oval than the other.

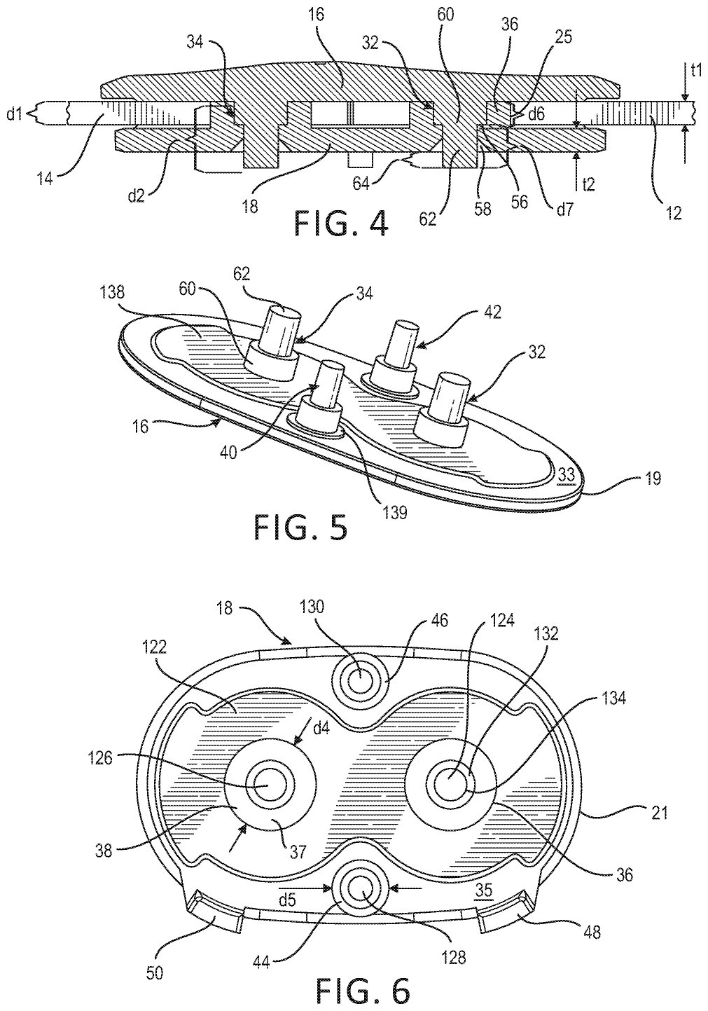

The outer cover plate may be of a larger oval than the inner cover plate, so the outside perimeter of the outer cover plate covers that of the inner cover plate. The width and/or length dimensions between the two may be within about 20%, 15%, 10%, or about 5% of one another. By non-limiting example, in an embodiment, the width of the inner cover plate may be about 33.5 mm, while the width of the outer cover plate may be about 36.5 mm. The length of the oval shape may similarly be larger for the outer cover plate relative to the inner cover plate, e.g., providing a small lip of the outer cover plate that extends beyond the perimeter of the inner cover plate, around the oval profile of the cover plates. Such a configuration is best apparent in FIGS. 4 and 6. The extension of the outer cover plate beyond the perimeter of the inner cover plate may be about 1.5 mm, around the entire oval perimeter. In such an embodiment, the width and length may each be about 3 mm shorter for the inner cover plate than the outer cover plate.

Indeed, from the foregoing, an advantage of molding the components of the hinge is that additional features or relative sizes can be prepared without detracting from the basic structure leading to the functionality of the hinge. Indeed, different polymeric materials can be used for the outer cover plate versus the inner cover plate, or different textures may be provided, such as a smoother outer cover plate to provide improved aesthetic appearance and features. Yet, the cover plates are still formed from compatible polymeric materials to enable bonding or pulse staking according to the foregoing examples. Moreover, the hinge arms may be formed from dissimilar materials, according to the application of hinge, such as bearing weight or requiring different strength tolerances.

Rather than forming the components of the polycentric hinge from numerous separate components (e.g., formed from metal materials), the present polycentric hinge comprises just two pieces that may be formed from a polymeric material, e.g., formed by injection molding or other suitable processes (e.g., machining, etc.). Where the two pieces (i.e., the inner and outer cover plates) are formed of such a polymer material, they may be welded together through an ultrasonic welding technique, rather than requiring joining through mechanical fasteners. In other embodiments, any other suitable technique may be employed for joining the two cover plates together (e.g., other welding techniques, use of an adhesive, etc.).

As described in further detail below, the inventors have discovered that the polycentric hinge comprised of just two pieces can be formed from a polymeric material (e.g., nylon) which may be welded together via pulse staking to provide strength and durability characteristics that are equal to or better than the current hinge configurations which employ numerous components, which components must be assembled.

The components are preferably formed from injection molded nylon reinforced with long carbon fibers. This specific composition of polymeric material may have a tensile strength of 250-300 MPa, a tensile modulus between 29000-30000 MPa, and a tensile elongation between 0.5-3%. Further the material may have a flexural strength of 400-450 MPa, a flexural modulus between 21000-22000 MPa, a notched Izod impact of 250-350 J/m, an un-notched Impact of 1000-1500 J/m, and a DTUL @ 1820 kPa between 225-275° C. The weight percent of the carbon fiber within the nylon may be between 30-50%, with a specific gravity in the range of 1.2-1.4. A benefit of using nylon in the construction of components is that it provides for a better friction material due to its ability to slide.

Other materials which can be injection molded, both with and without reinforcement fibers, are also possible to be used. All thermoplastic materials can be molded with such purpose if they meet the required strength. The ideal materials for the hinge are stiff and strong but not brittle. The hinge arms and cover plates do not need to be made of the same material. The hinge arms may be made of aluminum to comply with reimbursement codes needed to be malleable, but it is possible to use other materials like heat formable plastics.

U.S. Pat. No. 9,668,903 shows an exemplary orthopedic device configured as a knee brace, where the embodiments of the hinge of this disclosure may be connected to the upper and lower hinge arms, corresponding to upper and lower frame portions, respectively, of the knee brace as appreciated by those of skill in the art. The knee brace frame may take on many shapes, such as those shown and described in U.S. Pat. No. 5,230,697, granted Jul. 27, 1993, U.S. Pat. No. 8,048,013, granted Nov. 1, 2011, and U.S. Pat. No. 8,740,829, granted Jun. 3, 2014, each of which is incorporated by reference in its entirety. The hinge may be considered as comprising either just the inner and outer cover plates, or first and second hinge arms secured to one another by the inner and outer cover plates, or generally comprise frame portions having the equivalent of hinge arms secured together by the inner and outer cover plates.

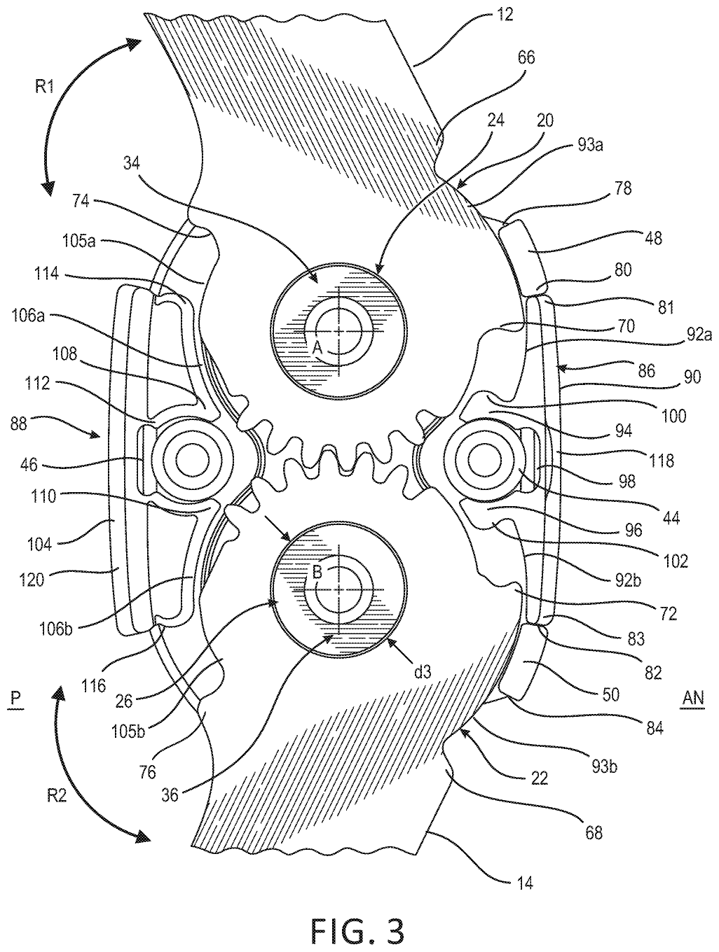

As shown in FIG. 3, rotation stops 86, 88 may secure to the third and fourth bearings 44, 46 to limit rotation of the hinge arms 12, 14 relative to one another, e.g., to prevent hyperextension of the knee joint (e.g., extension stop 86 may prevent rotation beyond) 180°. The extension stop 86 may be configured to prevent extension beyond any desired angle. The flexion stop 88 is provided to limit flexion to only any desired angle (e.g., 90°). Such a flexion stop may function similarly as described above relative to extension stop 86. The stops may be inserted from the anterior side AN of the hinge for extension control, and stops may be inserted from the posterior side P of the hinge for flexion control.

Various extension and flexion stops arranged at different extension, and flexion angles may be used, configured, and dimensioned accordingly. Aside from the specific description of how the stops are configured to secure to the third and fourth bearings, examples of providing flexion and extension stops configured for different rotational angles are described in further detail in U.S. Pat. No. 9,788,988, granted Oct. 17, 2017, incorporated herein by reference in its entirety.

The shape of the stops may differ depending on whether they are used as an extension stop or a flexion stop, and according to the angle or angles by which stops are intended to limit. The stops are preferably formed by a plastic material to offer resiliency for the prongs to snap-fit onto one of the bearings, and to allow them to be molded to shape, thereby offering a variety of stops with the hinge according to different angles (i.e., in angular increments such as at 5- or 10-degree changes).

The arrangement of the third and fourth bearings 44, 46 advantageously eliminates the need for any tool to secure the rotation stops to the hinge. Moreover, the hinge plates mitigate any need for additional structure by forming the bearings and corresponding protrusions. The injection-molded structure of the cover plates provides structure formed by the cover plates, which eliminates any need for miscellaneous extraneous components, thus leading to the conclusion that by injection molding the four parts, first and second cover plates 16, 18 and first and second hinge arms 12, 14, all their features can be provided among such four parts to create an inexpensive yet effective and functional polycentric hinge 10. Therefore, such an arrangement is a significant advantage over the prior art in that not only does the hinge properly function with a minimum of parts but is further arranged with a robust structure to accommodate a simplified configuration for attaching rotation stops without tools or fasteners.

Referring to the extension stop 86, and the flexion stop 88 in FIG. 3, the stops each have a pair of prongs 94, 96, 108, 110 with a clearance 98, 112 defined between interior surfaces of the pair of prongs 94, 96, 108, 110. The pairs of prongs 94, 96 and 108, 110 are sized and configured to removably snap-fit to at least one of the third and fourth bearings 44, 46. The prongs 94, 96, 108, 110 define first and second abutment surfaces 100, 102, 114, 116 on exterior surfaces opposite the clearance 98, 112. The first and second hinge arms 12, 14 form third and fourth stops 70, 72 of the anterior side AN and first and second projections 74, 76 of the posterior side P arranged to abut the first and second abutment surfaces 100, 102, 114, 116, and limit rotation of the first and second hinge arms 12, 14 in a first rotational direction R1, R2.

At least one of the first and second cover plates 16, 18, for example, the second cover plate 18 in FIG. 3, defines first and second tabs 48, 50 formed along the peripheral edge 21 and are the built-in 0° extension stops. Other stops may be added if different degrees of extension or flexion limitation are needed. The tabs form first stop surfaces 78, 84 arranged for abutment by first and second stops 66, 68 on the anterior side AN formed by the first and second hinge arms 12, 14 to limit a maximum extension of the hinge, such that as if there is no extension stop inserted and secured to the hinge along an anterior side AN of the hinge 10. The first and second tabs 48, 50 preferably protrude the predetermined distance d1 from the first or inner surface of the second cover plate and are located on opposite sides of the first rotation stop 86, 88. The first and second tabs 48, 50 form second stop surfaces 80, 82 arranged proximate or adjacent to first and second ends 81, 83 of the rotation stop 86.

The rotation stops 86, 88 define arcuate interior surfaces 92a, 92b, 106a, 106b arranged for a peripheral surface 93a, 93b, 105a, 105b of the first and second geared ends 20, 22 to rotated relative thereto without interference. The rotation stops 86, 88 preferably define a stop face 90, 104 arranged for blocking access into the hinge along a clearance formed between first and second cover plates 16, 18. The rotation stops 86, 88 each preferably define a peripheral flange 118, 120 having a thickness of the clearance or predetermined distance d1.

FIGS. 7-9 illustrate another embodiment of a hinge 140. The hinge 140 includes modified first and second cover plates 142, 144, and the first and second hinge arms 12, 14. The first and second cover plates 142, 144 are distinguished as having mating bosses or tubular bearings (demarcated by the dashed lines) 146, 152, each having the same or coequal height defined by distances d8, d9 corresponding to a thickness t1 of the hinge arms 12, 14. The bosses or tubular bearings 146, 152 extend from the corresponding bearing surfaces 158, 160. The coequal height has the advantage of limiting or removing warpage of the parts by evenly distributing the height of the bosses or tubular bearings, and evenly distributing pressure exerted by the hinge arms due to rotation about each of the first and second plates.

The first cover plate 142 has a base portion 148 that extends from the tubular bearing 146 and an extension 150 that extends from the base portion 148 for eventual welding to the second cover plate 144. The tubular bearing 152 extends from a thickness of the second cover plate 154, and has coaxial openings 154, 155 for receiving the base portion 148, and the extension 150, respectively, with the base portion 148 resting against seat 157 about the opening 155 of the second cover plate 144. The second cover plate 144 defines the outlet recess 156 through which the extension 150 extends, and into which molten material of the extension is received upon welding.

In observing FIG. 9, the second cover plate 144 has first and second bearing surfaces 160, 162 split by gate 170 defining a recess. The gate 170 is provided to minimize warping of the tubular bearings during manufacturing, particularly due to the usage of reinforcing fibers and the formation of the openings 154, 156. The gate 170 eliminates the tangling of fibers at the center of the second cover plate, and the possibility of forming a bump thereat.

A periphery of the opening 154 may be defined by alternating pattern 166 of recesses 168, such as in the depicted embodiment in FIG. 9. The alternating pattern 166 eliminates warpage at the tubular bearings by removing material whereat fibers may tangle during the formation of the tubular bearings, which still offers sufficient surface area to receive the base portion 148. The depicted image is only exemplary, and a variety of material-saving variations may be employed.

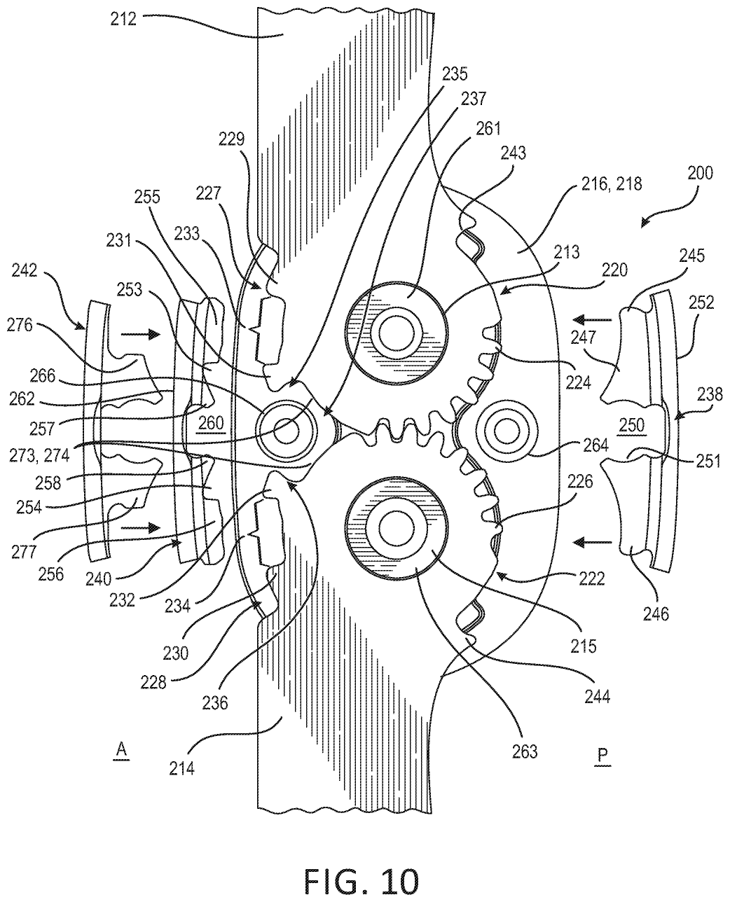

According to the disclosure, FIG. 10 is another embodiment 200 of a hinge, likewise formed from plastic. As with the embodiment of FIG. 1, the hinge 200 includes a first hinge arm 212 defining a first hole 213 at a first geared end 220 thereof, and a second hinge arm 214 defining a first hole 215 at a second geared end 222 thereof. However, unlike in the earlier embodiment, the hinge 200 is adapted to receive a flexion stop 240 arranged for 0 degrees flexion, and the hinge arms define features arranged to interlock with features of the flexion stop beyond those previously disclosed.

Specifically, at least part of the interlocking occurs because of the flexion stop 240 having at least one plug, particularly first and second plugs 255, 256, adapted to be received by at least one clearance, particularly first and second clearances 233, 234, defined by at least one of the first and second hinge arms 112, 114. Specifically, the first and second clearances 233, 234 are formed on anterior sides 227, 228 of the hinge at the first and second geared ends 220, 222, respectively.

As with the preceding embodiments, the hinge 200 includes first and second cover plates 216, 218. Either of the first or second cover plates can be arranged in the preceding embodiments and may have interchangeable components. For the sake of explanation in FIG. 10, the second cover plate 218 defines first and second bearings 261, 263 protruding from an interior first surface. The first and second holes 213, 215 of the first and second hinge arms 212, 214, respectively, are sized and configured to freely rotate about the first and second bearings 261, 263. The first and second holes 213, 215 are preferably coaxial to the first and second bearings 261, 263. Likewise, the second cover plate 218 defines third and fourth bearings 264, 266 located between and offset from the first and second bearings 261, 263. The third and fourth bearings 264, 266 and protrude from the second cover plate 218, and are arranged for flexion and extension stops 240, 242, to secure thereto, as in preceding embodiments.

The first and second geared ends 220, 222, are arranged to rotate about the first and second bearings 261, 263, and the first and second sets of teeth 224, 226, defined along part or a segment of the periphery of the circumference of the first and second geared ends 220, 222 mesh within one another about a range of motion of the hinge 200. The posterior side P of the first and second hinge arms 212, 214 define first and second projections 243, 244 adapted to abut corresponding first and second abutment surfaces 245, 246 of the flexion stop 238, when attached to the bearing 264.

As with the preceding flexion stop 88, the flexion stop 238 may be designated for different degrees of flexion. For example, while the depicted flexion stop 238 may be arranged for 30 degrees of rotation, the hinge 200 may include a set of flexion stops at various predetermined increments of rotation. Similarly, the flexion stop 238 inserts from the posterior side P of the hinge and defines a clearance 250 with an inner periphery 251 that can resiliently snap onto the bearing 264. The inner side of the flexion stop 238 may define surfaces 247 that do not interfere with rotation of the geared ends 220, 222 within the prescribed range of motion. The outer side of the flexion stop may be defined by a stop face 252 that limits intrusion into the hinge 200 of foreign objects and offer a surface by which a clinician or user can grasp the flexion stop for insertion and removal from the hinge.

The hinge 200 is arranged with the flexion stop 240 for the anterior side, in contrast to the flexion stop 238 defined for 30 degrees of flexion inserted from the posterior side. The flexion stop 240 is arranged for 0 degree of flexion, thereby placing the hinge 200 in full extension (i.e., 0 degree flexion). Unlike the corresponding extension stop 242, resembling extension stop 86, the flexion 240 engages and interlocks with features defined by the first and second geared profiles 220, 222.

The flexion stop 240 and the corresponding interlocking features defined by the first and second hinge arms 212, 214 provide a more stable locking arrangement, particularly as the hinge 200 is formed only by plastic. By offering interlocking components more robustly, than for example the flexion stop 238, the hinge can be maintained in 0 degrees flexion, without movement. While the other flexion and extension stops capably fulfill their intended purpose, it is more critical in 0 degrees flexion to withstand movement. The bolstering by interlocking features of the flexion stop 240 and hinge arms 212, 214 enable such more robust locking of the hinge from movement.

The first hinge arm 212 forms a first clearance 233 and the second hinge arm 114 forms a second clearance 234. The first flexion stop 240 defines first and second plugs 255, 256 adapted to fit in the first and second clearances 233, 234, respectively, to prevent rotation of the first hinge arm 212 relative to the second hinge arm 214. The first hinge arm 212 defines a first outer stop 229 and a first inner stop 231, bounding opposed ends of the first clearance 233 about a circumference of the first geared end 220. Similarly, the second hinge arm 214 defines a first outer stop 230 and a second inner stop 232 bounding opposed ends of the second clearance 234 about a circumference of the second geared end 222.

The first and second inner stops 231, 232 define a cavity 237 therebetween. In observing the extension stop 242, an outer profile of the prongs 276, 278 corresponds in shape to the cavity 237. The first and second inner stops 231, 232 define first and second biasing surfaces 235, 236 adapted to engage the prongs 276, 278. A periphery of the first and second geared ends 220, 222 generally extends circumferentially from the first and second inner stops 231, 232 to first and second sets of geared profile teeth 224, 226. A periphery of the cavity 237 is defined by segments 273, 274 of the first and second geared profiles 220, 222 devoid of geared profile teeth.

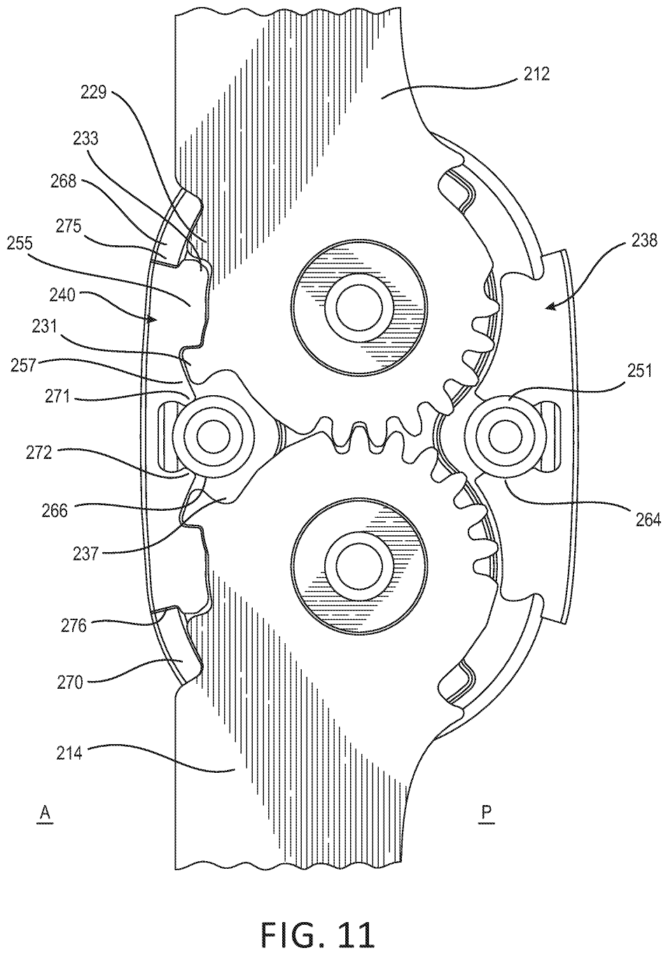

As depicted in FIG. 11, to further facilitate the interlocking of the components of the flexion stop 240 with the first and second hinge arms 212, 214, the flexion stop 240 defines a first groove 253 adapted to receive an end surface of the first inner stop 231. The first groove 253 is bordered by a first wedge 257 along which the first inner stop 231 rests. Similarly, the flexion stop 240 defines a second groove 254 adapted to receive an end surface of the second inner stop 232. The second groove 254 is bordered by a second wedge 258 along which the second inner stop 232 rests. The flexion stop 240 has first and second tips 271, 272 that abut the bearing 266, and a cavity 260 that may abut the bearing 266 to retain the flexion stop yet further 240 in place, and resist movement by the hinge arms, as flexion is locked. The flexion stop 240 may have an outer face 262 as in other embodiments.

Likewise, one of the first and second cover plates 216, 218 may have tabs 268, 270, as in preceding embodiments, and such tabs 268, 270, may further interlock with abutment faces 275, 276 of the flexion stop 240, yet further retaining the hinge in 0 degrees of flexion. Along with the outer face 262 and the tabs 268, 270, the anterior side of the hinge 200 may be enclosed, with the flexion stop 240 interlocking the components of the hinge to block access from the anterior side.

It is to be understood that not necessarily all objects or advantages may be achieved under an embodiment of the disclosure. Those skilled in the art will recognize that a hinge may be embodied or carried out, so it achieves or optimizes one advantage or group of advantages as taught herein without achieving other objects or advantages as taught or suggested herein.

The skilled artisan will recognize the interchangeability of various disclosed features. Besides the variations described, other known equivalents for each feature can be mixed and matched by one of ordinary skill in this art to build and use an orthopedic device under the principles of the present disclosure. Furthermore, the skilled artisan will understand that the features described may be adapted to other methods and types of orthopedic devices.

Although this disclosure describes certain exemplary embodiments and examples of a hinge, it will be understood by those skilled in the art that the present disclosure extends beyond the specifically disclosed components to other alternative embodiments and/or uses of the disclosure and obvious modifications and equivalents thereof. It is intended that the present disclosure should not be limited by the disclosed embodiments described above and may be extended to other applications that may employ the features described.

1. A hinge for an orthopedic device, comprising:
a first hinge arm defining a first hole at a first geared end thereof;
a second hinge arm defining a second hole at a second geared end thereof;
first and second cover plates for hingedly joining the first and second hinge arms;
wherein the first cover plate defines first and second protrusions extending from a bearing surface protruding from an interior first surface thereof, and the second cover plate defines first and second bearings protruding from an interior first surface of the second cover plate and arranged for receiving the first and second protrusions into openings defined thereby, the first and second bearings extending a predetermined distance at least a length at or greater than a thickness of the first and second hinge arms;
wherein the first and second holes of the first and second hinge arms, respectively, are sized and configured with a diameter at least a size of an outer diameter of the first and second bearings to freely rotate about the first and second bearings, the first and second holes being coaxial to the first and second bearings, the first and second protrusions extend coaxially relative to the first and second bearings and the first and second holes along first and second axes (A, B).

2. The hinge of claim 1, further comprising third and fourth bearings located between and offset from the first and second bearings, the third and fourth bearings being located proximate a peripheral edge of the second cover plate and protrude from the second cover plate and parallel to the first and second bearings.

3. The hinge of claim 2, wherein the third and fourth bearings have a length being identical as the predetermined distance of the first and second bearings.

4. The hinge of claim 3, wherein the third and fourth bearings define an outer diameter being smaller than the outer diameter of the first and second bearings.

5. The hinge of claim 2, wherein the first cover plate defines third and fourth protrusions arranged to extend into third and fourth openings defined by the third and fourth bearings along third and fourth axes (C, D).

6. The hinge of claim 1, wherein the interior first surface of the first cover plate is arranged to extend flush with a top surface of the first and second bearings, the first and second protrusions having a length greater than a combined length of the predetermined distance of the first and second bearings and a thickness of the second cover plate wherein an excess portion of the first and second protrusions extends beyond the second cover plate.

7. The hinge of claim 6, wherein the excess portion is arranged to be melted or sonically welded to fill an outlet recess formed along an exterior surface to secure the first cover plate to the second cover plate, the outlet recess depending from the first and second openings, and having an enlarged diameter relative thereto to accommodate the excess portion melted thereinto by welding or other appropriate techniques to form an integral connection of the first cover to the second cover wherein polymeric material from the first and second covers mix with one another to form a cohesive bond and material section.

8. The hinge of claim 6, wherein the first and second protrusions each form a base portion having a base width or diameter, and a base length, and an extension portion extending from the base portion, the base width or diameter being greater than an extension width or diameter, the extension portion including the excess portion, the base length generally corresponding to the predetermined distance, and the extension portion having a length exceeding the thickness of the second cover plate, wherein a total length of the protrusions is been shortened wherein the protrusions do not come to a stop, and the plates are arranged to come to a hard stop with the hinge arms.

9. The hinge of claim 8, wherein the first and second openings define an opening base portion configured and dimensioned to the base portion, and an opening extension portion is sized and configured to accommodate the width or diameter of the extension portion, the opening extension portion arranged shorter than the extension portion.

10. The hinge of claim 9, wherein the opening base portion is configured in a star shape to minimize warping and create more even wall thickness.

11. The hinge of claim 5, wherein the third and fourth protrusions are arranged similarly with a base portion and an extension portion, as in the first and second protrusions, and the third and fourth openings, including the third and fourth protrusions having an excess portion used to bond and form an integral connection of the first cover plate to the second cover plate.

12. The hinge of claim 2, further comprising at least a first rotation stop having a pair of prongs with a clearance defined between interior surfaces of the pair of prongs, the pair of prongs sized and configured to removably snap-fit to at least one of the third and fourth bearings.

13. The hinge of claim 12, wherein the pair of prongs define first and second abutment surfaces on exterior surfaces thereof opposite the clearance, the first and second hinge arms defining third and fourth stops of an anterior side of the hinge and first and second projections of a posterior side of the hinge and arranged to abut the first and second abutment surfaces, and limit rotation of the first and second hinge arms in a first direction.

14. The hinge of claim 13, wherein at least one of the first and second cover plates defines first and second tabs defined along the peripheral edge and have first stop surfaces arranged for abutment by first and second stops of the anterior side formed by the first and second hinge arms to limit a maximum extension of the hinge, wherein if there is no extension stop inserted and secured to the hinge along an anterior side of the hinge.

15. The hinge of claim 14, wherein the first and second tabs protrude the predetermined distance from the first surface and are located on opposite sides of the first rotation stop, the first and second tabs form second stop surfaces arranged proximate or adjacent to first and second ends of the first rotation stop.

16. A hinge for an orthopedic device, comprising:
a first hinge arm defining a first hole at a first geared end thereof;
a second hinge arm defining a second hole at a second geared end thereof;
a first flexion stop has at least one plug adapted to be received by a clearance defined by at least one of the first and second hinge arms.

17. The hinge of claim 16, wherein the first hinge arm forms a first clearance and the second hinge arm forms a second clearance, the first flexion stop forming first and second plugs adapted to fit in the first and second clearances, respectively, to prevent rotation of the first hinge arm relative to the second hinge arm.

18. The hinge of claim 17, wherein the first hinge arm defines a first outer stop and a first inner stop bounding opposed ends of the first clearance about a circumference of the first geared end.

19. The hinge of claim 18, wherein the second hinge arm defines a first outer stop and a second inner stop bounding opposed ends of the second clearance about a circumference of the second geared end;
wherein the first and second inner stops define a cavity therebetween, a periphery of the first and second geared ends extending generally circumferentially from the first and second inner stops to first and second sets of geared profile teeth, a periphery of the cavity being defined by a segments of the first and second geared profiles devoid of geared profile teeth.

20. The hinge of claim 18 wherein the first flexion stop defines a groove adapted to receive an end surface of the first inner stop, the groove bordered by a wedge along which the first inner stop rests;
wherein a second cover plate defines first and second bearings protruding from an interior first surface, wherein the first and second holes of the first and second hinge arms, respectively, the first and second holes are sized and configured to freely rotate about the first and second bearings, the first and second holes being coaxial to the first and second bearings;
wherein the second cover plate defines third and fourth bearings located between and offset from the first and second bearings, the third and fourth bearings and protrude from the second cover plate;
wherein the first and second cover plates for hingedly joining the first and second hinge arms.
